(19) United States
(12) Patent Application Publication
(10) Pub. No.: US 2009/0077695 A1
(43) Pub. Date: Mar. 19, 2009

(54) Interferon Produced In Plastids

(76) Inventor: Henry Daniell, Winter Park, FL (US)

Correspondence Address: ENRIQUE G. ESTEVEZ, 255 SOUTH ORANGE AVE, SUITE 1401, ORLANDO, FL, 32802-3791, US

(21) Appl. No.: 12/059,340

(22) Filed: Mar. 31, 2008

Related U.S. Application Data
(60) Provisional application No. 60/909,108, filed on Mar. 30, 2007.

(51) Int. Cl. A01H 5/00; C12N 1/00; C12N 5/04; C07K 14/00; C12N 15/00

(52) U.S. Cl. 800/298; 435/317.1; 435/419; 435/414; 530/351; 435/320.1

(57) ABSTRACT

Disclosed herein is an expression cassette containing a polynucleotide encoding an IFNα2b polypeptide and having two overlapping primers at a 5′ end encoding a polyhistidine tag and a thrombin cleavage site fused to the polypeptide, the expression cassette carried by a vector competent for integrating the expression cassette in a plastid genome. Also disclosed is a transgenic plastid, preferably a chloroplast, containing a genome transformed by integration of an expression cassette having a non-plant gene encoding an IFNα2b polypeptide and having regions that encode a polyhistidine tag and a thrombin cleavage site fused with the IFNα2b polypeptide.

### RELATED APPLICATIONS

This application claims priority from U.S. provisional application Ser. No. 60/909,108, which was filed on Mar. 30, 2007; this application is also a continuation in part of U.S. Ser. No. 11/406,522, which was filed on Apr. 18, 2006, and which is a continuation in part of U.S. Ser. No. 11/230,299 filed Sep. 19, 2005; which is a continuation of U.S. Ser. No. 09/807,742, filed Apr. 18, 2001, which claims priority to U.S. Ser. No. 60/185,987, filed Mar. 1, 2000, U.S. Ser. No. 60/263,473, filed Jan. 23, 2001 and U.S. Ser. No. 60/263,668, filed Jan. 23, 2001; Ser. No. 11/406,522 above is also a continuation in part U.S. Ser. No. 09/079,640 filed May 15, 1998; which claims priority to U.S. Ser. No. 60/055,413, filed Aug. 7, 1997 and U.S. Ser. No. 60/079,042, filed Mar. 23, 1998; all of these applications are incorporated herein by reference in their entirety including any figures, tables, or drawings.

### GOVERNMENT SUPPORT

The work leading to the presently disclosed invention was supported in part by U.S. government grants USDA 3611-21000-017-00D and NIH RO1 GM 63879 to Henry Daniell. Accordingly, the U.S. Government may have certain rights in the invention, as specified by law.

### FIELD OF THE INVENTION

The present invention relates to the field of molecular pharming and, more particularly, to plant-made human interferon α-2b (hereinafter IFNa2b).

### REFERENCES

Baron, S., Coppenhaver, D. H., Dianzani, F., Fleischmann, W. R. J., Hughes, T. K. J., Klimpel. G. R., Niesel, D. W., Stanton, G. J. and Tyring, S. K. E. (1992) Interferon: Principles and Medical Applications. Galveston, Tex.: University of Texas Medical Branch.
Belardelli, F., Ferrantini, M., Santini, S. M., Baccarini, S., Proietti, E. Colombo, M. P., Sprent, J. and Tough, D. F. (1998) The induction of in vivo proliferation of long-lived CD44hi CD8+ T cells after the injection of tumor cells expressing IFNalphal into syngeneic mice. Cancer Res., 58, 5795-5802.
Birch-Machin, I., Newell, C. A., Hibberd, J. M. and Gray. J. C. (2004) Accumulation of rotavirus VP6 protein in chloroplasts of transpiastomic tobacco is limited by protein stability. Plant Biotechnol. J., 2, 261-270.
Bodo, G. and Maurer-Fogy. I. (1986) Characterization of different molecular species in affinity purified recombinant human interferon alpha 2. In: The Interferon System (Dianzani, F. and Rossi, G. B. eds), pp 23-27. New York: Raven Press.
Brassard, D. L., Grace, M. J. and Bordens, R. W. (2002) Interferon-alpha as an immunotherapeutic protein. J. Leukoc. Biol., 71 565-581.
Chebolu, S, and Daniell, H. (2006) Stable expression of GAL/GALNAc lectin of Entamoeba histolytica in transgenic chloroplast and immunogenicity in mice towards vaccine development for amebiasis. Plant Biotechnol. J., in press.
Collins. G. B., Legg, P. D. and Kasperbauer, M. C. (1974) Tobacco hybrid LAMD-609. Crop Sci., 14, 72-80.
Cousens, L. P., Orange, J. S., Su, H. C. and Biron, C. A. (1997) Interferon-alpha/beta inhibition of interleukin 12 and interferon-gamma production in vitro and endogenously during viral infection. Proc. Natl. Acad. Sci. USA, 94. 634-639.
Cowley, G. (2002) Hepatitis C. The insidious spread of a killer virus. Newsweek, 139, 46-53.
Cramer, C. L., Boothe, J. G. and Oishi, K. K. (1999) Transgenic plants for therapeutic proteins: linking upstream and downstream strategies. Curr. Top. Microbiol. Immunol., 240, 95-118.
Daniell, H. (1997) Transformation and Foreign Gene Expression in Plants Mediated by Microprojectile Bombardment. In: Methods in Molecular Biology, pp 463-490. Humana Press.
Daniell, H. (2002) Molecular strategies for gene containment in transgenic crops. Nat. Biotechnol., 20, 581-586.
Daniell, H., Carmona-Sanchez. 0. and Burns, B. (2004a) Chloroplast derived antibodies. biopharmaceuticals and edible vaccines. In: Molecular Farming (Fischer, R. and Schillberg, S. eds), pp 113-133. Wiley-VCH Verlag.
Daniell, H., Datta, R., Varma, S., Gray, S, and Lee. S. B. (1998) Containment of herbicide resistance through genetic engineering of the chloroplast genome. Nat. Biotechnol., 16, 345-348.
Daniell. H., Kumar, S, and Dufourmantel. N. (2005a) Breakthrough in chloroplast genetic engineering of agronomically important crops. Trends Biotechnol., 23, 238-245.
Daniell, H., Lee. S.-B., Panchal, T. and Wiebe, P. O. (2001) Expression of the native cholera toxin B subunit gene and assembly as functional oligomers in transgenic tobacco chloroplasts. J. Mo/. Biol., 311, 1001.
Daniell, H., Ruiz, O. N. and Dhingra, A. (2004b) Chloroplast genetic engineering to improve agronomic traits. Methods Mo/. Biol., 286, 111-138.
Daniell, H. Ruiz, O. N. and Dhingra, A. (2005b) Chloroplast genetic engineering to improve agronomic traits. Methods Mo/. Biol., 286, 111-138.
De Maeyer, E. and De Maeyer-Guignard, J. (1988) Interferons and Other Regulatory Cytokines. New York: John Wiley & Sons.
de Waard-Siebinga, I., Creyghton, W. M., Kool, J. and Jager, M. J. (1995) Effects of interferon alfa and gamma on human uveal melanoma cells in vitro. Br. J. Opthalmol., 79, 847-855.
DeGray, G., Rajasekaran, K., Smith, F., Sanford, J. and Daniell, H. (2001) Expression of an Antimicrobial Peptide via the Chloroplast Genome to Control Phytopathogenic Bacteria and Fungi. Plant Physiol., 127, 852-862.
Edelbaum, 0., Stein, D., Holland, N., Gafni, Y., Livneh, 0., Novick, D., Rubinstein, M. and Sela, I. (1992) Expression of active human interferon-beta in transgenic plants. J. Interferon. Res., 12, 449-453.
Eibl, C., Zou, Z., Beck, A., Kim, M., Mullet, J. and Koop, H. U. (1999) In vivo analysis of plastid psbA, rbcL and rp132 UTR elements by chloroplast transformation: tobacco plastid gene expression is controlled by modulation of transcript levels and translation efficiency. Plant J., 19, 333-345.
Fernandez-San Milian, A., Mingo-Castel, A. Miller, M. and Daniell, H. (2003) A chloroplast transgenic approach to hyper-express and purify Human Serum Albumin, a protein highly susceptible to proteolytic degradation. Plant Biotechnol. J., 1, 71-79.
Fidler, I. J., Gersten, D. M. and Budmen, M. B. (1976) Characterization in vivo and in vitro of tumor cells selected for resistance to syngeneic lymphocyte-mediated cytotoxicity. Cancer Res., 36, 3160-3165.
Gidlund, M., Orn, A., Wigzell, H., Senik, A. and Gresser, I. (1978) Enhanced NK cell activity in mice injected with interferon and interferon inducers. Nature, 273, 759761
Glenz, K., Bouchon, B., Stehle, T. Wallich, R., Simon, M. M. and Warzecha, H. (2006) Production of a recombinant bacterial lipoprotein in higher plant chloroplasts. Nat. Biotechnol., 24, 76-77.
Gresser, I. Guy-Grand, D. Maury, C. and Maunoury, M. T. (1981) Interferon induces peripheral lymphadenopathy in mice. J. Immunol., 127, 1569-1575.
Grevich, J. J. and Daniell, H. (2005) Chloroplast genetic engineering: Recent advances and future perspectives. Crit. Rev. Plant Sci., 24, 83-107.
Gutterman, J. U. (1994) Cytokine therapeutics: lessons from interferon alpha. Proc. Natl. Acad. ScL USA, 91, 1198-1205.
Gwynne, D. I. O., CA), Buxton, F. P. O., CA), Pickett, M. H. O., CA), Davies, R. W. O., CA) and Scazzocchio, C. B. s. Y., FR) (1993) Vectors in use in filamentous fungi, United States: Gist-Brocades N. V. (Delft, NL).
Henco, K., Brosius, J., Fujisawa, A., Fujisawa, J. I., Haynes, J. R., Hochstadt, J., Kovacic, T., Pasek, M., Schambock, A., Schmid, J., Todokoro, K., W6lchli, M.,
Nagata, S, and Weissmann, C. (1985) Structural relationship of human interferon alpha genes and pseudogenes. J. Mol. Biol., 185, 227-260.
Horn, M. E., Woodard, S. L. and Howard, J. A. (2004) Plant molecular farming: systems and products. Plant Cell Rep., 22, 711-720.
Ishikawa, R. and Biron, C. A. (1993) IFN induction and associated changes in splenic leukocyte distribution. J. Immunol., 150, 3713-3727.
Kamarajugadda, S, and Daniell, H. (2006) Chloroplast-derived anthrax and other vaccine antigens: their immunogenic and immunoprotective properties. Expert Rev. Vaccines, 5, 839-849.
Koya, V., Moayeri, M., Leppla, S. H. and Daniell, H. (2005) Plant-Based Vaccine: Mice Immunized with Chloroplast-Derived Anthrax Protective Antigen Survive Anthrax Lethal Toxin Challenge. Infect. Immun., 73, 8266-8274.
Kumar, S, and Daniell, H. (2004) Engineering the chloroplast genome for hyperexpression of human therapeutic proteins and vaccine antigens. Methods Mol. Biol., 267, 365-383.
Leelavathi, S, and Reddy. V. S. (2003) Chloroplast expression of His-tagged GUS-fusions: a general strategy to overproduce and purify foreign proteins using transplastomic plants as bioreactors. Mol. Breeding, 11, 49.
Limaye, A., Koya, V., Samsam, M. and Daniell, H. (2006) Receptor-mediated oral delivery of a bioencapsulated green fluorescent protein expressed in transgenic chloroplasts into the mouse circulatory system. Faseb J., 20, 959-961.
Maeda, S., Kawai, T., Obinata, M., Fujiwara, H., Horiuchi, T., Saeki, Y. Sato, Y. and Furusawa, M. (1985) Production of human alpha-interferon in silkworm using a baculovirus vector. Nature, 315, 592-594.
Mayfield, S. P., Franklin, S. E. and Lerner, R. A. (2003) Expression and assembly of a fully active antibody in algae. Proc. Natl. Acad. ScL USA, 100, 438-442.
McBride, K. E., Svab, Z., Schaaf, D. J., Hogan, P. S., Stalker, D. M. and Maliga, P. (1995) Amplification of a chimeric Bacillus gene in chloroplasts leads to an extraordinary level of an insecticidal protein in tobacco. Biotechnology (N Y), 13, 362-365.
Molina, A., Hervas-Stubbs, S. Daniell, H., Mingo-Castel, A. M. and Veramendi, J. (2004) High-yield expression of a viral peptide animal vaccine in transgenic tobacco chloroplasts. Plant Biotechnol. J., 2, 141-153.
Ohya, K. Matsumura, T., Ohashi, K., Onuma, M. and Sugimoto, C. (2001) Expression of two subtypes of human IFN-alpha in transgenic potato plants. J. Interf. Cytok. Res. 21, 595-602.
Pestka, S., Langer, J. A. Zoon, K. C. and Samuel, C. E. (1987) Interferons and their actions. Annu. Rev. Biochem., 56, 727-777.
Quesada-Vargas, T., Ruiz, O. N. and Daniell, H. (2005) Characterization of heterologous multigene operons in transgenic chloroplasts: transcription, processing, and translation. Plant Physiol., 138. 1746-1762.
Rubinstein, S., Familletti, P. C. and Pestka, S. (1981) Convenient assay for interferons. J. Virol. 37, 755-758.
Ruhiman, T., Ahangari, R., Devine. A. L., Samsam, M. and Daniell, H. (2007) Expression of cholera toxin B—proinsulin fusion protein in lettuce and tobacco chloroplasts—oral administration protects against development of insulitis in non-obese diabetic mice. Plant Biotechnol J, Revised manuscript in review.
Ruiz, O. N. and Daniell, H. (2005) Engineering cytoplasmic male sterility via the chloroplast genome by expression of {beta}-ketothiolase. Plant Physiol., 138, 1232-1246.
Sambrook, J., Fritsch, E. F. and Maniatis, T. (1989) Molecular Cloning—A Laboratory Manual, 2nd Edition. New York: Cold Spring Harbor Laboratory Press.
Senik, A., Gresser, I., Maury, C., Gidlund, M., Orn. A. and Wigzell, H. (1979) Enhancement by interferon of natural killer cell activity in mice. Cell Immunol., 44, 186-200.
Slocombe, P., Easton, A., Boseley, P. and Burke, D. C. (1982) High-level expression of an interferon alpha 2 gene cloned in phage M13mp7 and subsequent purification with a monoclonal antibody. Proc. Natl. Acad. Sci. USA, 79, 5455-5459.
Staub, J. M., Garcia, B., Graves, J., Hajdukiewicz, P. T., Hunter, P., Nehra, N., Paradkar, V., Schlittler, M., Carroll, J. A., Spatola, L., Ward, D., Ye, G. and Russell, D. A. (2000) High-yield production of a human therapeutic protein in tobacco chloroplasts. Nat. Biotechnol., 18, 333-338.
Stuart-Harris, R. P., R. D. (Eds.) (1997) Clinical Applications of the Interferons. London: Chapman & Hall Medical.
Swaminathan, S, and Khanna, N. (1999) Affinity purification of recombinant interferon alpha on a mimetic ligand adsorbent. Protein Expr. Purif., 15, 236-242.
Tregoning, J. S., Nixon, P., Kuroda, H., Svab, Z., Clare, S., Bowe, F., Fairweather, N., Ytterberg, J., van Wijk, K. J., Dougan, G. and Maliga, P. (2003) Expression of tetanus toxin Fragment C in tobacco chloroplasts. Nucleic Acids Res., 31, 1174-1179.
Trinchieri, G. (1989) Biology of natural killer cells. Adv. Immunol., 47, 187-376. Venkataraman, N. Cole, A. L. Svoboda, P., Pohl, J. and Cole, A. M. 2005) Cationic Polypeptides Are Required for Anti-HIV-1 Activity of Human Vaginal Fluid. J. Immunol., 175, 7560-7567.
Vilcek, J. and Sen, G. C. (1996) Interferons and other cytokines. In: Fundamental Virology (Fields, B. N., Knipe, D. M. and Howley, P. M. eds). New York: Lippincott-Raven Publishers.
Wang, W. (1999) Instability, stabilization, and formulation of liquid protein pharmaceuticals. Int. J. Pharm., 185, 129-188.
Watson, J., Koya, V., Leppla. S. H. and Daniell, H. (2004) Expression of Bacillus anthracis protective antigen in transgenic chloroplasts of tobacco, a nonfood/feed crop. Vaccine, 22, 4374-4384.
Wei, X., Decker, J. M., Liu, H. Zhang, Z., Arani, R. B., Kilby, J. M., Saag. M. S., Wu. X. Shaw, G. M. and Kappes, J. C. (2002) Emergence of resistant human immunodeficiency virus type 1 in patients receiving fusion inhibitor (T-20) monotherapy. Antimicrob. Agents Chemother., 46, 1896-1905.
Zhu, Z. Hughes, K. W., Huang, L., Sun, B., Liu, C. and Li, Y. (2004) Expression of human alpha-interferon cDNA in transgenic rice plants. Plant Cell Tiss. Org., 36, 197-204.

### BACKGROUND OF THE INVENTION

The type I interferons (IFNs) are part of the body's first line of defense against not only viral attack, but also invasion from bacterial pathogens, parasites, tumor cells and allogeneic cells from grafts (De Maeyer and De Maeyer-Guignard, 1988). IFNs alpha and beta display significant amino acid sequence homology (30%) and bind to the same receptor (Pestka et al., 1987). Members of the IFN-a family were the first to be highly purified, sequenced, cloned and produced by recombinant DNA (Henco et al., 1985). Although they are known for their inhibition of viral replication (Vilcek and Sen, 1996). IFN-a is also involved in regulating cytokine and cytokine receptor gene expression (Cousens et al. 1997), mediating cellular proliferation and differentiation (Baron et al. 1992), modifying immune cell distribution (Gresser et al., 1981; Ishikawa and Biron, 1993) and activating natural killer (NK) cell cytotoxic activity (Trinchieri, 1989). In fact, IFN-a is a potent inhibitor of cell growth (Pestka et al., 1987). Perhaps most importantly, IFN-a may be a critical link between the innate and adaptive immune responses (Brassard et al., 2002).

Recombinant interferon alpha 2b (IFN-a2b) was first approved by the Food and Drug Administration (FDA) in 1986 for the treatment of hairy cell leukemia. Today, it is routinely administered for the treatment of various cancers and viral diseases, but the cost of treatment is prohibitive in many developing countries (Stuart-Harris, 1997). The average cost of treatment using the marketed recombinant IFN-a2b is $26,000 for a twelve-month course in the United States (Cowley, 2002).

The current therapeutic IFN-a2b is marketed under the names PEG-Intron™ and IntronA® and is made in the microbial system Escherichia coli. Many eukaryotic proteins cannot be expressed in prokaryotic hosts because the required formation of two disulfide bonds (Bodo and Maurer-Fogy, 1986) presents a major limitation, adding to the cost of production of the mature, biologically active IFN-a2b. As a result, IFN-a2b tends to aggregate to form inclusion bodies that need to be solubilized, renatured and refolded into an active protein (Swaminathan and Khanna, 1999). Other production platforms, such as silkworm using baculovirus (Maeda et al., 1985) or phage vectors (Slocombe et al. 1982) have been used to express IFN-a2b, but at low levels that are not competitive with the current system.

The plant nuclear genome has been used to express human therapeutic proteins for more than a decade (Horn et al., 2004). Expression of IFN-a2b and IFN-a8 has been achieved by transforming potato nuclei (Ohya et al., 2001). In tobacco only 0.000017% fresh weight of transgenic leaves contained IFN-b (Edelbaum et al. 1992). In addition, negligible amounts of IFN-a were produced in nuclear-transformed rice (Zhu et al., 2004). The low and variable expression levels (due to position effect and gene silencing) using nuclear transformation have been less than adequate to be commercially feasible, in addition to problems of transgene containment that arise due to field planting. Therefore, strategies are required to increase expression levels in plants and to facilitate transgene containment, allowing rapid and less expensive production of therapeutic proteins.

The chloroplast expression system is quite versatile in producing proteins as small as 20 amino acids, e.g.—magainin (DeGray et al., 2001), or as large as 135 kDa. e.g.—Cry 1Ac (McBride et al. 1995). Plastids can produce monomeric (Fernandez-San Milian et al., 2003; Staub et al., 2000) or multimeric proteins, including cholera toxin B (CTB) (Daniell et al. 2001), CTB fusion proteins (Limaye et al., 2006), and Guy's 13 (Daniell et al., 2004a) and herpes simplex virus (Mayfield et al., 2003) antibodies. Therapeutic proteins with 2-17 disulfide bonds, including human serum albumin (Fernandez-San Milian et al., 2003) and somatotropin (Staub et al., 2000), have already been produced in transgenic chloroplasts, with appropriate post-translational modifications, including lipid modifications, e.g.—OspA lipoprotein (Glenz et al., 2006). The chloroplast expression system has also been used to produce fully functional vaccine antigens against bacterial pathogens, including cholera (Daniell et al., 2001), anthrax (Koya et al., 2005; Watson et al., 2004) and tetanus (Tregoning et al., 2003); viral pathogens, including canine parvovirus (Molina et al., 2004) and rotavirus VP6 (Birch-Machin et al., 2004); and protozoan pathogens, including amoeba (Chebolu and Daniell, 2006). The proper folding, disulfide bond formation and functionality of chloroplast-derived vaccine antigens and therapeutic proteins have been demonstrated by several assays including macrophage lysis, GM1-ganglioside binding, systemic immune responses, protection against pathogen or toxin challenge and growth or inhibition of cell cultures (Daniell et al., 2004a; Kamarajugadda and Daniell, 2006). Multiple genes have been engineered via the chloroplast genome in a single transformation event, facilitating the synthesis and assembly of multicomponent vaccines (Quesada-Vargas et al., 2005). Therefore, any therapeutic protein, irrespective of size, can be made with desired post-translational modifications, with the notable exception of glycosylation (Daniell et al., 2004a: Daniell et al., 2005a). However, a few foreign proteins have been highly unstable within transgenic chloroplasts. For example, interferon gamma was degraded within chloroplasts, showing <0.2% total soluble protein (TSP), but expression of up to 7% TSP was achieved via fusion with GUS (Leelavathi and Reddy, 2003). Human insulin was similarly unstable in transgenic chloroplasts; fusion with CTB resulted in high level expression (up to 16% TSP) and facilitated oral delivery studies to achieve protection against the development of insulitis in non-obese diabetic mice (Ruhlman et al., 2007). Such N-terminal degradation is not unique to chloroplasts. Presently, all commercially produced insulin in bacteria or yeast is produced as a fusion protein; when expressed without fusion, insulin is rapidly degraded.

Plant chloroplasts are ideal bioreactors, and it has been shown that one acre of chloroplast transgenic plants can produce up to 360 million doses of clean, safe and fully functional anthrax vaccine antigen (Koya et al., 2005; Watson et al., 2004). This is a relatively rapid system for scale-up because a single tobacco plant (a non-food, non-feed crop) produces up to one million seeds, which is adequate to plant more than 100 acres (8,000 plants/acre and 40 metric tons of leaf biomass/acre). Transgenes integrated into the chloroplast genome are maternally inherited in most crops, ensuring they are not spread via pollen and thereby offering transgene containment (Daniell, 2002; Daniell et al., 1998; Grevich and Daniell, 2005). Alternatively, cytoplasmic male sterility could be engineered via the chloroplast genome, thereby eliminating the production of viable pollen (Ruiz and Daniell, 2005). An additional advantage is that transformed seeds can be stored indefinitely and vaccine antigens can be produced on demand, eliminating the need to stockpile vaccine antigens under low temperature and with limited shelf life.

Type I interferons inhibit viral replication and cell growth and enhance the immune response, and therefore have been shown to have many clinical applications. IFN-a2b ranks third in world market use for a biopharmaceutical, behind only insulin and erythropoietin. The average annual cost of IFN-a2b for the treatment of hepatitis C infection is $26,000, and is therefore unavailable to the majority of patients in developing countries.

### SUMMARY OF THE INVENTION

With the foregoing in mind, the present invention advantageously provides expression of interferon and, more specifically, IFN-a2b in transgenic plastids, particularly in tobacco chloroplasts (cpIFN-a2b), and demonstrates the functionality of purified IFN-a2b using commercial interferons as comparative standards.

FN-a2b was expressed in tobacco chloroplasts and transgenic lines were grown in the field after obtaining USDA-APHIS approval. Stable, site-specific integration of transgenes into chloroplast genomes and homoplasmy was confirmed through several generations. IFN-a2b levels reached up to 20% of total soluble protein or 3 mg per g of leaf (fresh weight). Transgenic IFN-a2b had similar in vitro biological activity to commercially produced PEG-Intron™ when tested for its ability to protect cells against cytopathic viral replication in the standard VSV CPE assay and to inhibit early stage HIV infection. The antitumor and immunomodulating properties of IFN-a2b were also seen in vivo. Chloroplast-derived IFN-a2b increased the expression of MHC I on splenocytes and the total number of NK cells. Finally, IFN-a2b purified from chloroplast transgenic lines (cpIFN-a2b) protected mice from a highly metastatic tumor line. This demonstration of high levels of expression of IFN-a2b, transgene containment, and biological activity akin to that of commercial preparations of IFN-a2b facilitated the first field production of a plant-derived human blood protein, a critical step towards human clinical trials and commercialization.

Accordingly, the invention discloses an expression cassette containing a polynucleotide encoding an IFNα2b polypeptide and having two overlapping primers at a 5′ end encoding a polyhistidine tag and a thrombin cleavage site fused to the polypeptide, the expression cassette carried by a vector competent for integrating the expression cassette in a plastid genome.

The invention further includes a transgenic plastid, preferably a chloroplast, containing a genome transformed by integration of an expression cassette having a non-plant gene encoding an IFNα2b polypeptide and having two overlapping primers at a 5′ end that encode a polyhistidine tag and a thrombin cleavage site fused with the IFNα2b polypeptide.

Yet an additional aspect of the present invention includes a plant homoplasmic for a plastid or chloroplast genome transformed by integration of an expression cassette having a gene encoding an IFNα2b polypeptide and having two overlapping primers at a 5′ end that encode a polyhistidine tag and a thrombin cleavage site fused with the IFNα2b polypeptide.

### BRIEF DESCRIPTION OF THE DRAWINGS

Some of the features, advantages, and benefits of the present invention having been stated, others will become apparent as the description proceeds when taken in conjunction with the accompanying drawings, presented for solely for exemplary purposes and not with intent to limit the invention thereto, and in which:

FIG. 1, according to an embodiment of the present invention, shows confirmation of chloroplast integration and determination of homoplasmy/heteroplasmy in To generation of both varieties. (a) The 0.81 kb DNA probe containing chloroplast flanking sequences; (b) DNA fragments of 7.9 kb indicate no transformed chloroplast and DNA fragments of 9.9 kb are observed when the chloroplast genome has the transgenes integrated; lanes 1,5: untransformed wild type; lanes 2-4: Transgenic LAMD-609 lines; lanes 6-9: transgenic Petit Havana lines; (c) Western blot of LAMD-609 transgenic plants expressing cpIFN-a2b; low nicotine tissue extract separated on 15% SDS-PAGE with cpIFN-a2b detected by mouse monoclonal antibody against human IFN-a; lane 1: 80 ng PEG-Intron standard; lane 2: protein marker; lane 3: wild-type (untransformed) LAMD-609; lanes 4-7: transgenic LAMD609 lines expressing monomers and multimers of cpIFN-a2b; (d) Western blot of Petit Havana transgenic plants expressing cpIFN-a2b; Petit Havana leaf extract separated on 15% SDS-PAGE with cpIFN-c_x2b detected by mouse monoclonal antibody against human IFN-a; lane 1: 38 ng of IntronA; lane 2: protein marker; lane 3: 190 ng of IntronA; Lane 4: wild-type (untransformed) Petit Havana; lanes 5-7: transgenic Petit Havana lines expressing monomers and multimers of cpIFN-a2b; lanes 8-9: E. coli-transformed with IFN-a2b;

FIG. 2 shows quantitation of cpIFN-a2b; crude extracts of transgenic leaf material were analyzed for transgene expression by indirect ELISA and quantitation of transgenic IFNa2b was performed on the T1 generations of (a) and (b) LAMD and (c) and (d) Petit Havana plants; (a) and (c) show quantitation of cpIFN-a2b as a percentage of TSP; (c) and (d) show quantitation of cpIFN-a2b in mg transgenic protein per g fresh leaf weight; two different standards were used in ELISA: recombinant IFN-a2b with a covalent conjugate of monomethoxy polyethylene glycol (PEG-Intron) and recombinant human IFN-a2b; “Y” are young leaves (top few in the plant); “M” are mature leaves (fully developed); “O” are old leaves (bottom senescent leaves with decreased pigments); duration of illumination: 0, 1, 3 and 5 days of continuous illumination; error bars represent the standard error of the mean;

FIG. 3 shows that transgenic tobacco plants produce IFN-a2b with biological activity in vitro; (a) shows that transgenic IFN-a2b inhibits VSV-induced cytopathicity; BHK cells were pretreated with the indicated samples for 24 hours prior to VSV exposure; at the conclusion of the assay, cells were washed and stained with crystal violet for microscopic examination; in (b) and (c) transgenic IFN-a2b inhibits HIV infection, as determined by reduction in luciferase expression; TZM-BL cells were pretreated for 24 hours with the indicated reagents, and then exposed to two different HIV isolates; in (b) see the CCR5-tropic strain BaL, and in (c) the CXCR4-tropic strain IIIB; all treatments were performed in triplicate; error bars represent the standard error of the mean and asterisks indicate p values <0.05;

FIG. 4 presents immunohistochemical detection of the interferon response in mouse spleens; (a) provides representative photomicrographs of mouse spleen tissue; the tissues were fixed and 10 μm sections were stained either for the NK cell markers CD49b and NK1.1 or MHC I; for NK cell marker staining, the channels were read individually and also overlaid; note that because of the natural expression profile of MHC I, all cells are presumed to be positive; therefore, these data are designed to evaluate relative differences between the conditions; isotype controls depict the background fluorescence of the native tissue; photographs were taken with a 20× objective and are representative of more than 100 sections of each treatment condition; (b) and (c) show analysis of the mean fluorescence intensity (MFI) of splenocytes stained for MHC cells (b) and for NK cells (c); all tissue samples were acquired with equivalent MFI; error bars in (b) and (c) represent the standard error of the mean; asterisks indicate statistical comparisons between PEG-Intron and PBS, and purified cpIFN-a2b and PBS, with significant p values <0.05;

FIG. 5 shows phenotypic analysis of splenocytes; total splenocytes isolated from the various groups of mice in this study were analyzed by flow cytometry for surface marker expression; (a) shows representative flow cytometry plots indicating splenocyte expression of MHC I and CD49b; quadrant positions are based on isotype controls; (b) is a comparison of the MFI of MHC I across the various treatment groups; all cells comprising the MHC I+ population (i.e., both CD49b+ and CD49b″ populations) are represented; (c) is a comparison of expression of NK cell markers (CD49b and NK1.1) across the various treatment groups; data in (b) and (c) represent the means of each treatment group; error bars represent the standard error of the mean; asterisks indicate statistical comparisons between PEG-Intron and PBS; and purified cpIFN-a2b and PBS, with significant p values <0.05.

FIG. 6. shows that transgenic tobacco plants produce IFN-a2b with biological activity in vivo; (a) photomicrographs of lungs of mice injected with the metastatic tumor B16-F10; mice were treated via intraperitoneal injection as indicated for three consecutive days; (b) shows mean color intensity of tumor metastases; photomicrographs of lung samples were analyzed for color intensity; all samples were photographed with equal exposure settings; a calculation of total color was then achieved by selecting only those pixels that corresponded to the lung tissue; the mean color intensity was calculated for all samples of a particular group; the lower the intensity, the greater amount of black (tumor) in the image; error bars represent the standard error of the mean; asterisks indicate statistical comparisons between PEG-Intron and PBS, and purified cpIFN-a2b and PBS, with significant p values <0.05; and

FIG. 7 in the top panel shows a tobacco field ideal for the production of biopharmaceutical proteins; the middle and bottom panels show, respectively, in vitro antiviral response and in vivo immune responses in mouse spleens to chloroplast derived interferon.

### DETAILED DESCRIPTION OF THE PREFERRED EMBODIMENT

The present invention will now be described more fully hereinafter with reference to the accompanying drawings, in which preferred embodiments of the invention are shown. Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. Any publications, patent applications, patents, or other references mentioned herein are incorporated by reference in their entirety. In case of conflict, the present specification, including any definitions, will control. In addition, the materials, methods and examples given are illustrative in nature only and not intended to be limiting. Accordingly, this invention may, however, be embodied in many different forms and should not be construed as limited to the illustrated embodiments set forth herein. Rather, these illustrated embodiments are provided so that this disclosure will be thorough and complete, and will fully convey the scope of the invention to those skilled in the art. Other features and advantages of the invention will be apparent from the following detailed description, and from the claims.

### Results and Discussion

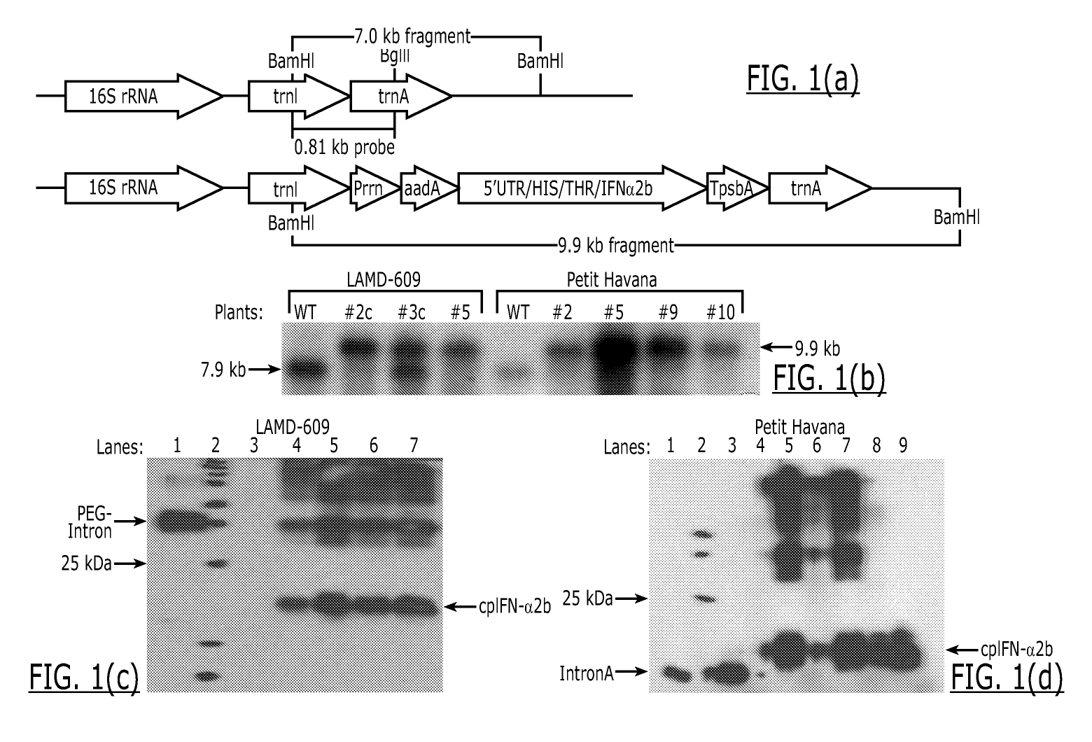

The 5.9 kb universal chloroplast vector (Daniell et al., 2004b) used in this study contains several unique features that facilitate the integration of cloned DNA into the plastid genome. This vector is as disclosed in U.S. application Ser. No. 09/079,640, now U.S. Pat. No. 7,129,391, which is incorporated herein by reference in its entirety. This process occurs exclusively through site-specific homologous recombination, thereby excluding the foreign vector DNA and enhancing transgene expression. The pLD-CtV vector incorporates the trnA and trnI genes (chloroplast transfer RNA transcripts coding for alanine and isoleucine, respectively) from the inverted repeat region of the tobacco chloroplast genome as flanking sequences for homologous recombination (Daniell et al., 2004b). This vector was successfully used herein as the backbone for insertion of the expression cassette 5′UTR/HIS/THR/IFNa2b cassette (FIG. 1a).

Selection and regeneration of transgenic lines. From 7 bombarded Petit Havana leaves, 27 green shoots appeared after 4 weeks. From 8 bombarded LAMD-609 leaves, 12 green shoots appeared within 7 weeks, indicating the shoots from the low-nicotine tobacco LAMD-609 took longer to sprout and were less numerous. Untransformed cells appeared bleached on the antibiotic because they did not contain the aadA gene. For the second round of selection, the Petit Havana shoots were able to thrive on 500 μg/ml of spectinomycin, while the LAMD-609 transformants only grew when the antibiotic concentration was lowered to 350 μg/ml spectinomycin.

Confirmation of homoplasmy. For both tobacco varieties, Southern blots were performed utilizing appropriate DNA probes to confirm integration of the IFNa2b cassette. A 0.81 kb DNA fragment containing the chloroplast trnA and trnI flanking sequences was used to probe total leaf DNA to determine homoplasmy or heteroplasmy after bombardment with pLD-RF-IFNa2b (FIG. 1a). This determination was also used to estimate chloroplast genome copy number. BamHI-digested DNA from transformed plants produced a 9.9 kb fragment when probed (FIG. 1 b). Untransformed plant DNA from both tobacco varieties produced a single 7.9 kb fragment, indicating no integration of foreign DNA. All but one transgenic line exhibited only the 9.9 kb fragment (FIG. 1 b), indicating homoplasmy; plant #3c had both the 7.9 kb and 9.9 kb fragments, indicating heteroplasmy. Attainment of homoplasmy in transformants provides a potential integrated transgene copy number of 10,000 copies per tobacco leaf cell (100 chloroplasts per plant cell×100 genomes per chloroplast) and indicates that homoplasmy can be achieved in the T₀ generation. Transgene integration and maintenance of homoplasmy has been demonstrated in subsequent generations (T₁, T₃, etc.; data not shown).

Characterization of cpIFNa2b. IFN-a2b is functional in its monomeric form, but does have a tendency to aggregate (Wang, 1999). For LAMD-609, monomers of cpIFN-a2b protein were detected at approximately 21.5 kDa (F. 1c), due to the presence of the polyhistidine tag and thrombin cleavage site and smaller than the PEG-Intron standard (approximately 32 kDa). For Petit Havana, monomers of cpIFN-a2b protein were also detected at approximately 21.5 kDa (FIG. 1d), slightly larger than the 19.2 kDa size of the IntronA standard. In addition, dimers and multimers of cpIFN-a2b were observed in both LAMD-609 and Petit Havana.

Those of skill in the art will recognize that due to its high proteolytic specificity, a thrombin cleavage site is a valuable biochemical tool. The amino acid sequence of a thrombin cleavage site has been previously published and is known to those of skill in the art. A thrombin cleavage site is useful following purification of the fusion protein, since thrombin can then be used to selectively cleave off the histidine tag from the interferon with a high degree of specificity.

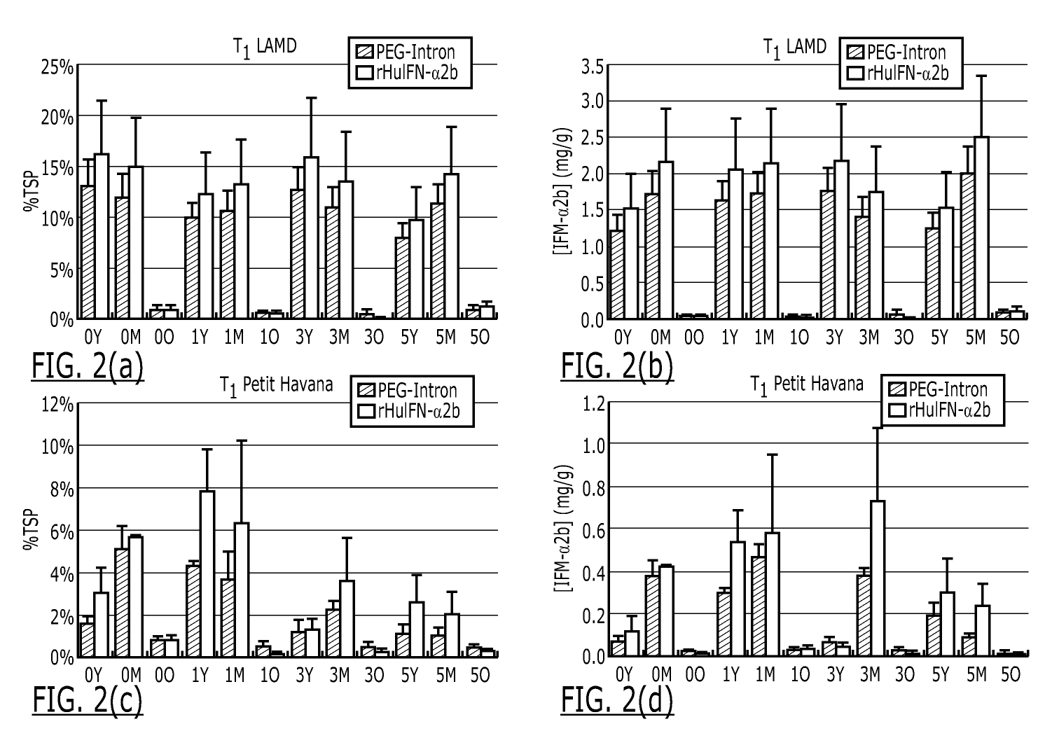

The physical condition of the plants, age and external factors such as illumination and changes in temperature in the growth chamber and greenhouse all contribute to the levels of protein expressed. ELISA was used to analyze how cpIFN-a2b levels varied in the young (Y), mature (M) and old (O) leaves of the same transgenic plants of Petit Havana and LAMD, as well as under different durations of illumination, using PEG-Intron and rHu-IFN-a2b as standards. In all transgenic lines grown in the greenhouse, the highest levels of cpIFN-a2b expression were obtained when compared to rHu-IFN-a2b as the standard (FIG. 2). Old leaves had the lowest level of expression, which corresponds to senescence inducing high proteolytic activity. Both young and mature leaves showed high levels of IFN-a2b expression, ranging from 1-3 mg/g fresh weight, or 8-21% TSP, in T₁ LAMD transgenic lines (FIGS. 2a and 2b). In Petit Havana transgenic lines, IFNa2b expression ranged from 0.2-1.2 mg/g fresh weight or 2-14% TSP (FIGS. 2c and 2d). The variation in expression levels may be due to a decrease in cpIFN-a2b levels upon continuous illumination; in LAMD transgenic lines, such illumination increased IFN-a2b accumulation. This highlights the difference between Petit Havana and LAMD in tolerating continuous light. The differences in protein levels correlate to differences in light intensity since the same source of nutrition was provided to all transgenic lines. It is also known that 5′ untranslated regions of the psbA gene located upstream of IFNa2b cassette enhance translation of the psbA gene in light (Eibl et al., 1999). The dramatic difference in the IFN-a2b levels in the greenhouse grown LAMD plants is most likely due to the light regulated translational enhancement by the psbA regulatory sequences.

Expression of cpIFN-a2b in field-grown biomass. In addition to the greenhouse, plants grown in the field were also examined. Approximately 2.5 months after transplantation in October, the biomass on stalks were cut 15 cm above ground with a forage harvester (Hege, Eging am See, Germany). From a single harvest, 107.7 kg of biomass was collected, shipped and stored in a commercial freezer (−20° C.) prior to analysis and purification. The plants had an average height of 43.2 cm and weighed 64 g. A single acre of tobacco biomass can be harvested 4-6 times within a growing season. As a result, there is the potential to generate greater than one metric ton of fresh biomass per acre under these conditions. As expected, Petit Havana plants had less height and weight per plant, and therefore less biomass, per acre than commercial cultivars, which can generate up to 40 times more per acre (Cramer et al. 1999). Based on 0.8 mg IFNa2b per gram of leaf biomass, 107.7 kg of harvested biomass contained about 87.2 grams of IFN-a2b. The limited yield was due to a single harvest of biomass at a very early age due to premature flowering of Petit Havana in the field. Lower expression of IFN-a2b was anticipated in young leaves because the psbA regulatory sequence used to express IFN-a2b is developmentally regulated and optimal levels of expression is achieved only in mature leaves of fully grown plants (Daniell et al., 2004a; Fernandez-San Milian et al., 2003; Koya et al., 2005; Limaye et al. 2006; Watson et al., 2004).

The present invention enabled production of 137.13 mg of IFN-a2b from 100 g of biomass at maturity in the greenhouse (Table 1); based on observed expression levels, this should yield 5.5×10⁷ mg of IFN-a2b in an acre of tobacco plants. A weekly dose of PEG-Intron, which is the E. coli-produced recombinant IFN-a2b treatment for hepatitis C, is 70 μg for an average 70 kg male (Schering Corp). Based on a 50% purification efficiency, approximately 400 million weekly doses for the treatment of hepatitis C could be produced in just one acre by the present invention.

Functional evaluation of cpIFN-a2b using VSV. First, cpIFN-a2b activity was evaluated by its antiviral properties. The activity of IFN-a2b is typically assayed by measuring its ability to inhibit the in vitro replication of the vesicular stomatitis virus (VSV) (Rubinstein et al., 1981). Staining cells with crystal violet at the end of the assay enabled us to visualize the degree of

TABLE 1
Yield of cpIFN-α2b in LAMD transonic
lines grown in the greenhouse.

| | | | Average | IFN-α2b | IFN-α2b per age |
|---|---|---|---|---|---|
| | Leaves | IFN-α2b | weight | in fresh | group (mg) |
| Leaf | per | per leaf | per leaf | leaf | (# leaves × [IFN- |
| age | plant | (mg) | (g) | (mg/g) | α2b] per leaf) |
| Young | 3.20 | 3.50 | 2.50 | 1.40 | 11.20 |
| Mature | 7.80 | 16.00 | 8.00 | 2.00 | 124.80 |
| Old | 4.50 | 0.25 | 5.00 | 0.05 | 1.13 |

| | |
|---|---|
| Total amount of recombinant IFN-α2b per plant | 137.13 |

Young leaves were among the top 2-3; old leaves were the bottom 2-3 that were senescent; and mature leaves were selected from the middle of the plant. CpIFN-α2b levels were quantified by ELISA.

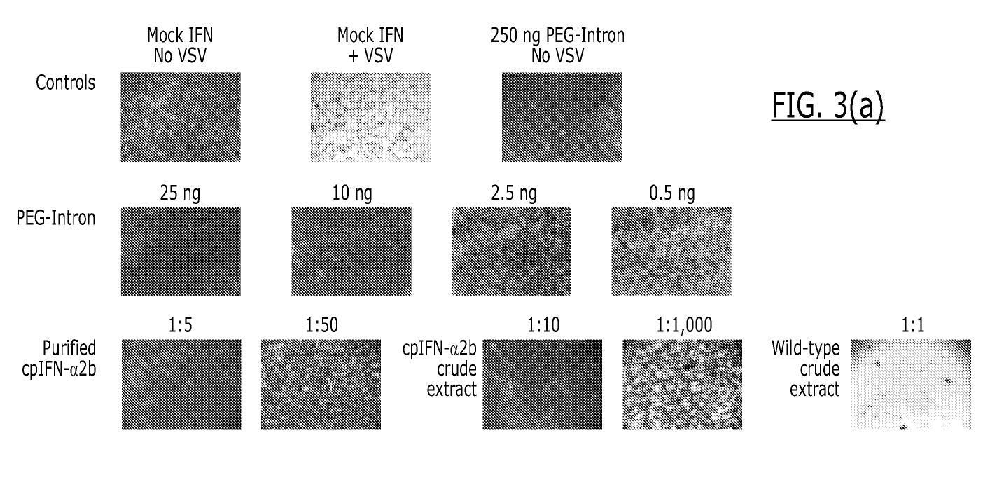

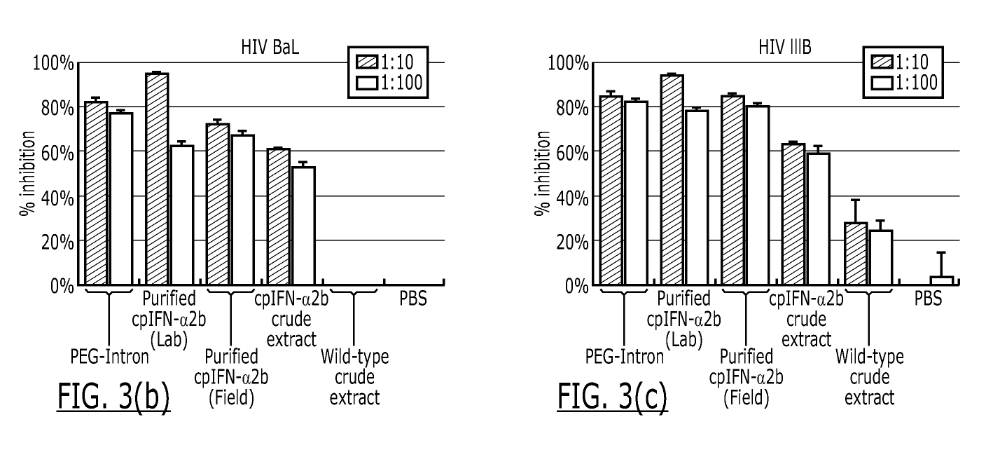

cytopathology under the microscope. Representative photomicrographs are shown in FIG. 3a. The mock controls—VSV alone and media alone—were assigned protection values of 0% (complete cytopathology) and 100% (complete protection), respectively. To rule out any toxic effects, IFN-a2b was tested in the absence of virus. As can be seen by the example of PEG-Intron, cells grew normally in the presence of up to tenfold higher concentrations of IFN-a2b (FIG. 3a). All experimental preparations of cpIFN-a2b were protective for cells, and did so in a dose-dependent manner. Purified cpIFN-a2b protected cells even when diluted 500-fold (data not shown). A crude extract of transgenic leaf material was still protective at the 1,000-fold dilution (FIG. 3A). Crude extracts of nontransgenic tobacco leaves were not protective at any dilution, indicating that it is the presence of transgenic IFN-a2b in the plant that mediates the protection.

The crystal violet staining also enabled us to quantitate the biological activity of the various IFN-a2b preparations tested. The activity is expressed in international units (IU) and was calculated by comparing levels of protection with controls. CpIFN-a2b samples from both lab and field sources were strongly protective (Table 2), as were their corresponding crude extracts. The weekly dose of PEG-Intron is also depicted in the table, to illustrate the levels

TABLE 2
Biological activity of IFN-α2b preparations. BHK cells were pre-treated
with the indicated samples for 24 hours prior to VSV exposure.
Concentrations were determined by comparing the results of each
experimental condition with the performance of PEG-Intron, the positive
control. These data represent the mean of four independent experiments,
with each treatment performed in duplicate.

| Sample | Concentration (IU/ml)¹ |
|---|---|
| Purified cpIFN-α2b (Lab sample) | 7.7 × 10⁷ |
| Purified cpIFN-α2b (Field sample) | 1.3 × 10⁸ |
| cpIFN-α2b crude extract (Lab sample) | 1.3 × 10⁶ |
| cpIFN-α2b crude extract (Field sample) | 2.8 × 10⁶ |
| Wild-type crude extract | 4.5 × 10⁰ |
| PEG-Intron (maximum weekly dose) | 1.1 × 10⁶ |

¹IU—International units, calculated by comparison with a leukocyte reference standard obtained from the NIAID Repository.
²The maximum weekly dose is based on the manufacturer's specified doseage and is calculated for a 70 kg individual.

of activity achievable via the transgenic chloroplast system. As can be seen, 100 mg of transgenic leaf material expressing IFN-a2b has approximately the same biological activity as one weekly dose of PEG-Intron.

Functional evaluation of cpIFN-a2b using HIV. To further investigate the antiviral properties of cpIFN-a2b, the protection of cells from HIV-1 entry and integration was investigated. The targets for infection in this assay are TZM-BL cells, a derivative of HeLa cells that express CD4, CCR5 and CXCR4, rendering them excellent targets for infection by both tropic varieties of HIV. These cells also contain a luciferase expression cassette that is driven by the HIV LTR. Thus, when HIV productively infects TZM-BL, the high levels of Tat produced drive transcription by the endogenous LTR, resulting in quantifiable luciferase expression.

Two different HIV isolates were tested: the CCR5-tropic strain BaL and the CXCR4-tropic strain IIIB. PEG-Intron demonstrated approximately 80% inhibition of luciferase activity at both dilutions tested (FIGS. 3b and 3c). Cp1FN-a2b from lab-derived plants showed nearly 100% protection from infection by both HIV strains at the 10-fold dilution. At the 100-fold dilution, protection from HIV BaL was nearly 60% (FIG. 3b) and from HIV IIIB, it was better than 80% (FIG. 3c). CpIFN-a2b from field-derived plants also demonstrated strong protection, better than 65% for both dilutions and both strains tested.

Protection by the transgenic plant crude extract was also noted. For HIV BaL, better than 50% protection was observed; and for HIV IIIB, better than 67% protection. Wild-type crude extract did not protect cells at all from infection by HIV BaL, but did provide minimal protection (<20%) from HIV IIIB, indicating that it is the IFN-a2b expressed by transgenic leaves that provides the bulk of specific protection.

In summary, it was observed that both forms of cpIFN-a2b tested—purified and crude extract from both lab and field sources—were protective in the in vitro antiviral assays. The successful performance of the transgenic plant crude extracts in the in vitro assays, with strong biological activity of cpIFN-a2b, demonstrates the advantage of higher levels of expression observed in chloroplast transgenic lines. After performing initial in vitro studies using plant crude extracts, all subsequent investigations were conducted with cpIFN-a2b: observed results were very similar to currently available commercial sources of interferon.

In vivo studies. Among the myriad effects IFN-a2b is reported to impart, up-regulation of MHC I molecules (de Waard-Siebing a et al., 1995) and activation of NK cells (Gidlund et al., 1978; Senik et al., 1979) comprise a major component of its antiviral and antitumor properties. Our in vivo assays were designed to address whether our preparations of cpIFN-a2b were similarly capable of regulating the immune response.

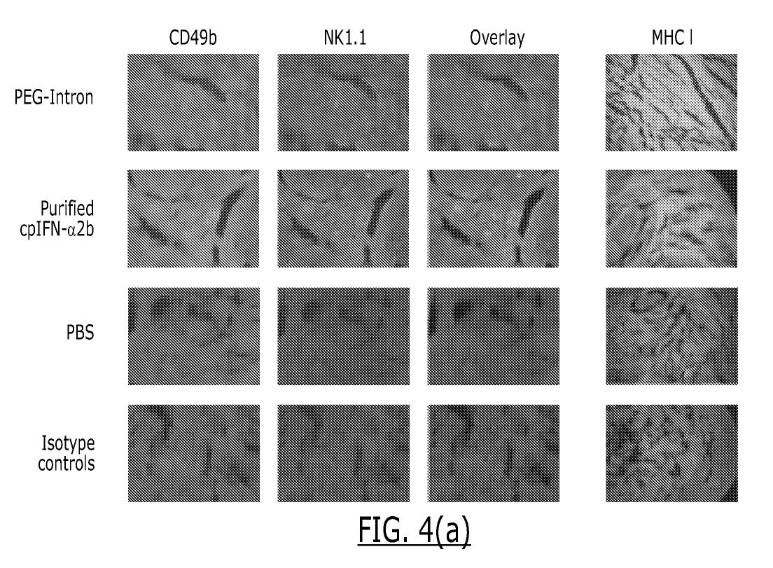

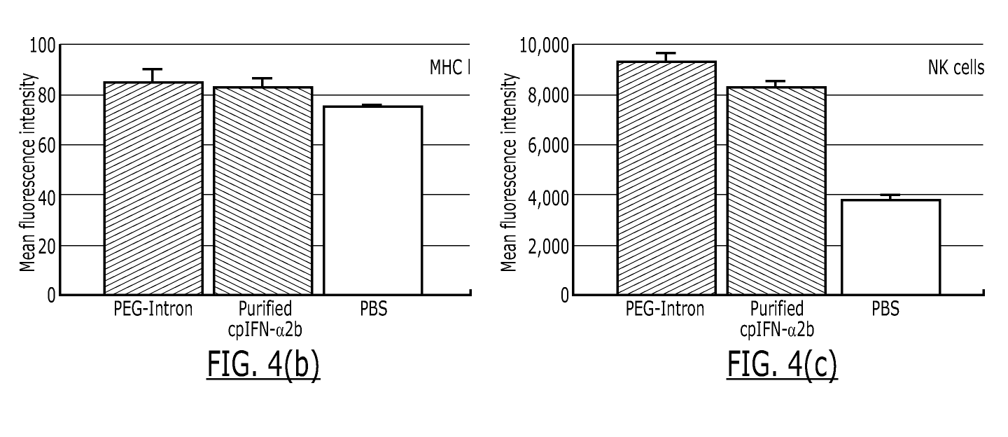

Cp1FN-cab up-regulates MHC I expression on splenocytes. Blind screens of more than 100 sections of spleen tissue from each treatment condition were performed for the expression of cell surface markers. The expression of MHC I on splenocytes was analyzed first. Because all nucleated cells express MHC I antigens, interested focused on the relative differences between the treatment groups. The basal level of MHC I expression of mouse spleen tissue can be seen in the PBS treatment condition (FIG. 4a). Treatment with cpIFN-a2b increased MHC I expression to levels similar to those achieved by PEG-Intron treatment. These results were confirmed by quantifying the immunofluorescence emissions data. Spleen tissue isolated from PBS-treated mice demonstrated a MFI of MHC I expression of 74.7 units (FIG. 4b). Mice treated with PEG-Intron had a significantly higher MFI (88.02 units; p=0.04). Mice treated with cpIFN-a2b also had a significantly higher MFI (83.27 units; p=0.003).

CpIFN-a2b increases the number of NK cells in vivo. We also screened spleen tissue for the presence of NK cells. In PBS-treated mice, we found little staining of cells positive for the NK cell markers CD49b and NK1.1 (FIG. 4a). In contrast, we detected more positive NK cell staining in mice treated with cpIFN-a2b. When we quantified the fluorescence emissions of the overlaid images, spleen tissue isolated from mice treated with PBS demonstrated a combined MFI (MFIcD49b×MFINKii) of 3,797 units (FIG. 4c). Mice treated with PEG-Intron had a significantly higher combined MFI of 9,366 units (p=1.6×10-9). Mice treated with cpIFN-a2b also had a significantly higher combined MFI value of 8,284 units (p=8.9×10-15).

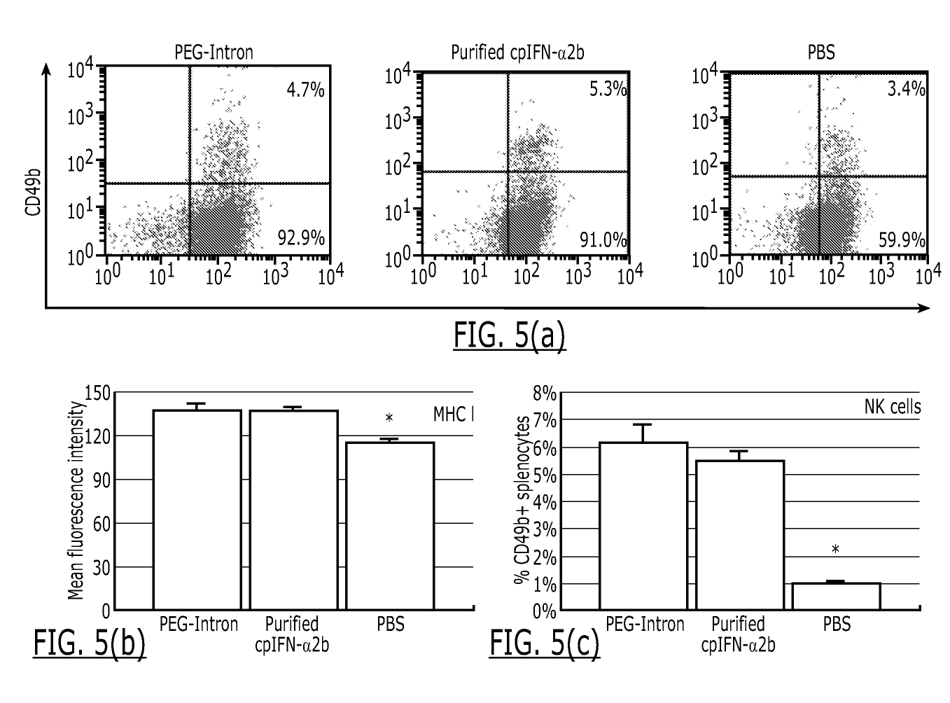

Finally, we analyzed the splenocytes by flow cytometry. Representative plots are depicted in FIG. 5a. Splenocytes isolated from mice treated with either PEG-Intron or cpIFN-a2b stained nearly 90% positive for MHC I expression (data not shown) and more than 6% were positive for expression of the NK cell marker CD49b (FIG. 5b). Mice treated with PBS had significantly fewer splenocytes staining positive for CD49b (p=0.04). In contrast, mice treated with cpIFN-a2b had nearly 5.5% of splenocytes staining positive for CD49b, also a significant difference (p=1.3×10-5). In addition, the MHC I+ cell population from mice treated with the cpIFN-a2b preparations had a nearly 20% greater MFI than that of PBS-treated mice (FIG. 5c), a significant difference (PEGIntron: p=0.01; cpIFN-a2b: p=0.03). Taken together, these data lend support to the notion that cpIFN-a2b possesses the biological function of up-regulating MHC I expression and NK cell markers.

CpIFN-a2b inhibits tumor metastasis in vivo. IFN-a has well-characterized antitumor activity in malignancies (Belardelli et al., 1998; Gutterman, 1994). We assessed the antitumor properties of cpIFN-a2b against B16-F10 cells, an aggressive metastatic melanoma cell line (Fidler et al. 1976).

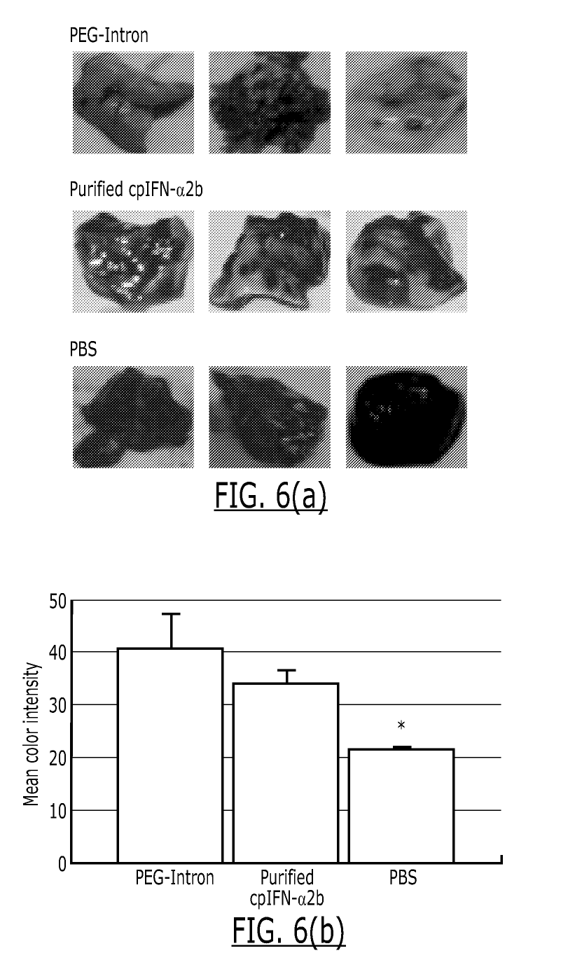

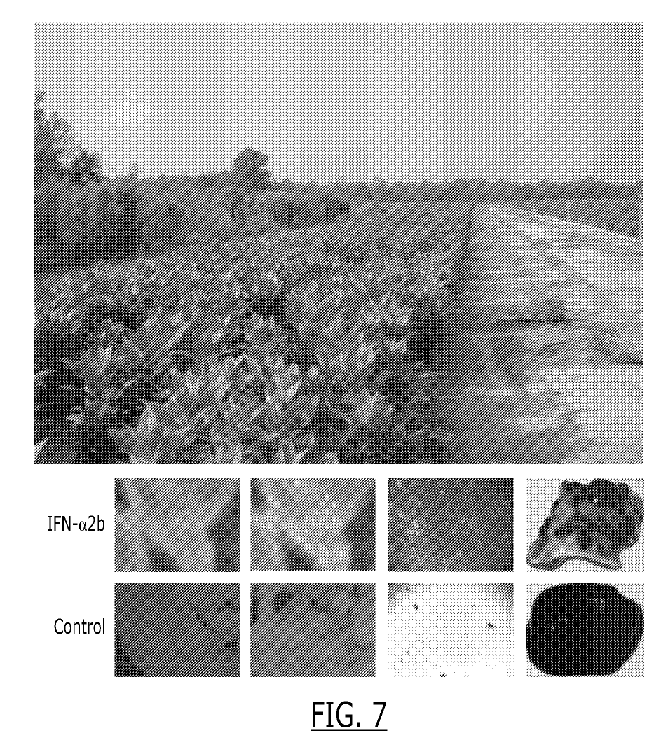

The degree of tumor metastasis could be empirically determined by macroscopic observation of the lungs after sacrifice (FIG. 6a). Mice treated with PBS had the highest degree of metastasis, indicated by near complete coverage of the lung with tumor. In contrast, mice treated with either PEG-Intron or cpIFN-a2b exhibited quantifiably fewer black spots (FIG. 6a). We analyzed these data further by calculating the color intensity of the images, designed to help quantitate the relative intensity of tumor (black) on a background of healthy lung tissue (red/pink). The calculations are summarized in FIG. 6b, where the mean color intensity (MCI) of each group is plotted. As expected, mice treated with the positive control preparation-PEG-Intron had a mean MCI of 40.82 units, significantly higher than that of the animals treated with PBS (21.58 units; p=0.02). CpIFN-a2b also significantly reduced the tumor metastasis to a mean MCI of 34.24 units (p=1.1×107). These data suggest that treatment of mice with cpIFN-a2b mediates antitumor activity and helps protect mice from the metastatic effects of the B16-F10 tumor line. Once again, as in previous assays. the performance of cpIFN-a2b is comparable to that of its commercial counterpart.

### Experimental Procedures

Construction of the pLD-RF-IFNa2b vector. The IFNa2b gene was purchased from American Type Culture Collection (Gwynne et al., 1993) (ATCC #53371; Manassas. Va.) in E. coli strain JM83 K-12. Sequencing results confirmed that the fragment in the pGL2BIFN vector was IFNa2b, the sequence of which has been previously reported and is known to those skilled in the art. Two overlapping primers were used at the 5′ end of the IFNa2b gene to include a thrombin cleavage site and a polyhistidine tag, with the reverse primer containing a NotI restriction site for further subcloning. After confirmation of the DNA sequence, the gel-eluted PCR product was ligated into a Bluescript vector containing the 5′ untranslated region (UTR) of the psbA gene. This fragment (5′UTR/HIS/THRUFNa2b) was inserted into the universal chloroplast vector pLD to create pLD-RF-IFNa2b. The recombinant DNA techniques were carried out as detailed previously (Sambrook et al., 1989).

Bombardment and selection of transgenic shoots. The Bio-Rad PDS-1000/He biolistic device, a particle delivery system, was used to bombard tobacco leaves (Daniell et al., 2005b; Kumar and Daniell, 2004). Two varieties of tobacco (Nicotiana tabacum) were generated for the bombardment: Petit Havana and LAMD-609 (low nicotine hybrid produced by backcrossing a Maryland type variety, MD-609, to a low nicotine-producing burley variety, LA Burley 21) (Collins et al., 1974). After recovering in the dark for 48 hours, leaves were placed in a laminar flow hood, were cut into 5 mm pieces and the pieces were placed on RMOP plates containing 500 μg/ml of spectinomycin for the first round of selection for transformants (Daniell, 1997; Daniell et al., 2004b). Approximately 4 weeks later, shoots growing from the original pieces were cut into 2 mm² pieces and transferred to fresh RMOP plates containing spectinomycin for a second round of selection. During this round of selection, the shoots that appeared were tested for cassette integration into the chloroplast genome by PCR analysis. Finally, after 4 weeks of secondary selection, the shoots were transferred to sterile jars containing fresh MSO medium with 500 μg/ml spectinomycin (Daniell et al., 2005b; Kumar and Daniell, 2004). When the shoots grew to fill the jars, the transgenic lines were transferred to pots with soil containing no antibiotic. Potted plants were grown in a 16-hour light/8-hour dark photoperiod in the growth chamber at 26° C. or in the greenhouse or field.

Southern blot analysis. Total plant DNA was extracted from transgenic T₀. plants and untransformed tobacco plants using the DNeasy Plant Mini Kit (Qiagen, Valencia, Calif.). Total plant DNA was digested with HincII and was probed by the flanking sequence probe, which was obtained from the pUC-Ct vector by digesting with BamHI and Bg/II to obtain a 0.81 kb fragment (FIG. 1a). The probe was prepared by random primed ³²P-labeling (Ready-To-Go DNA labeling beads, Amersham Biosciences, Pittsburgh, Pa.). The probes were hybridized to the membrane using the Quick-hyb solution and protocol (Stratagene, La Jolla, Calif.) as described previously (Daniell et al., 2005b; Kumar and Daniell, 2004). The radiolabeled blots were exposed to x-ray films and then developed in the x-ray film processor.

Immunoblot analysis. Plant extraction buffer (PEB) was made fresh on the same day as the Western blot analysis and contains 100 mM NaCl, 10 mM EDTA (pH 8), 200 mM Tris-HCl (pH 8), 0.05% Tween-20®, 0.1% SDS, 14 mM 3-mercaptoethanol (BME), 400 mM sucrose and 2 mM phenyl methyl sulfonyl fluoride (PMSF), which specifically inhibits serine proteases such as chymotrypsin, trypsin, and thrombin. From each transgenic Petit Havana and LAMD-609 line, leaf sections were cut and labeled as old (bottom), mature (middle), and young (top) leaves. Leaf material (100 mg) was ground in liquid nitrogen in cold, autoclaved mortars and pestles. PEB (200 ELI) was added to each plant sample on ice and then mixed in a Vortex® mixer for 10 seconds. A mouse anti-human IFN-a antibody (Abcam, Cambridge, Mass.) was used for the immunoblot analysis of the extracted plant proteins.

ELISA. Plant tissues were ground in liquid nitrogen using sterile mortars and pestles and then placed on ice. Plant protein extraction buffer (500 of 15 mM Na2CO3, 35 mM NaHCO3, 3 mM NaN3, 0.1% Tween-20®, pH 9.6) was added to each sample and briefly mixed in a Vortex® mixer. The samples were centrifuged at 5,000 rpm for 2 minutes and then placed back on ice. The supernatant was passed through a 0.22 μm filter and transferred to a fresh tube. Dilutions of crude extract ranging from 1:5 to 1:5,000 were made in coating buffer (15 mM Na₂CO₃, 35 mM NaHCO₃, 3 mM NaN₃, pH 9.6). PEG-Intron (a covalent conjugate of recombinant IFN-a2b with monomethoxy polyethylene glycol: Schering, Kenilworth, N.J.) was used as the standard and was also diluted in coating buffer. Aliquots of 100 μl of the diluted plant protein extract and standards were pipetted into a 96-well microtiter EIA plate in duplicate. The plate was covered with Parafilm® and incubated at room temperature for 4 hours. Wells were washed three times with 0.1% Tween-20® qq in phosphate-buffered saline (PBS-T), followed by three washes with deionized water. The plate was patted dry on paper towels, without letting the wells go completely dry. To detect the presence of IFN-a2b in each sample, 100 μl of mouse monoclonal antibody against human interferon (clone MMHA-2, PBL Biomedical, Piscataway. N.J.), diluted 1:2,500 in PBS supplemented with 3% non-fat, powdered milk (Carnation) and 0.1% Tween-20® (P-T-M) was added to each well and incubated for 1 hour at 37° C. After the incubation, the wells were washed as above, and 100 μl of the secondary antibody-goat anti-mouse IgG conjugated to horseradish peroxidase (American Qualex, San Clemente, Calif.) diluted 1:5,000 in P-T-M-was added to the wells and incubated for 1 hour at 37° C. After the incubation, the wells were washed as above and 100 μl of 3.3′,5,5′-tetramethylbenzidine (TMB) substrate (American Qualex) was added to the wells. After allowing 5 minutes for color change, 100 μl of 2M sulfuric acid was added to stop the reaction. The plate was immediately read on a microtiter plate reader (BioTek Instruments, Winooski, Vt.) using a 450 nm filter.

Estimation of total soluble protein. Total soluble protein (TSP) of plant crude extract was determined by Bradford assay. Bovine serum albumin (BSA, Sigma Chemical, St. Louis, Mo.) was used as a standard in concentrations ranging from 0.05 to 0.5 mg/ml. Aliquots of 10 μl of diluted plant extract and each standard were added in duplicate to wells of a 96-well microtiter plate (Cellstar, Greiner. Nurtingen, Germany). Bradford reagent (Bio-Rad Protein Assay. Bio-Rad, Hercules, Calif.) was diluted 1:4 with distilled water, filtered, and 200 μl was added to each well and assayed as previously described (Daniell et al., 2005b; Kumar and Daniell, 2004). The absorbance was read at 595 nm.

Field production of biomass expressing IFN-a2b. IFN seeds in Nicotiana tabacum cv ‘Petit Havana’ were initially propagated in the greenhouse using standard tobacco production practices for clipping, fertilizer and pesticide application suitable for field transplantation 30 days after seeding. The field was prepared prior to transplantation by the application of 138 kg/A ammonium nitrate using a Gandy® Drop Spreader and incorporated into the first 13 cm of soil using a John Deere® Soil Finisher. In August, seedlings were transplanted using a mechanical transplanter to non-raised beds at 40-cm row and 30-cm plant spacings. The mean height of the transplanted seedlings was 15 cm, with an average of 5 leaves each. and planted about 10 cm deep. Approximately 0.26 acre containing 7,369 plants was seeded. At the time of transplantation, the field was prepared by applying insecticides—456 g/A of Orthene® (Monsanto, St. Louis, Mo.) and 43 g/A Admire® (Bayer CropScience, Research Triangle Park, N.C.)—and herbicides—231 g/A Spartan® (Chemical Products Technologies, Cartersville, Ga.) and 922 g/A of Command® (FMC, Philadelphia, Pa.). Crop maintenance required application of the fungicides Quadris® at 228 g/A (Syngenta Crop Protection, Greensboro, N.C.), and Acrobat MZ® at 48 g/A (BASF, Research Triangle Park, N.C.), every 20-30 days to control blue mold infestation, and a single application of Orthene® at 456 g/A to control aphid infestation.

In vitro assays to study functionality of interferon Cells. Baby hamster kidney (BHK) and YAC-1 cells were obtained from ATCC (Manassas, Va.). H9. PM1, and TZM-BL cells were obtained from the National Institutes of Health AIDS Research and Reference Reagent Program (Germantown, Md.). BHK and TZM-BL cells were cultured in high-glucose DMEM supplemented with 10% heat-inactivated fetal bovine serum (FBS). H9 cells were cultured in RPMI 1640 supplemented with 10% FBS and 100 mM HEPES. PM1 cells were maintained at a density of 4-8×10⁵ cells/ml in RPMI 1640 supplemented with 20% FBS and 100 mM HEPES. YAC-1 cells were cultured in RPM! 1640 supplemented with 10% FBS, 2 mM L-glutamine, 10 mM HEPES, 1 mM sodium pyruvate, 4.5 g/L glucose and 1.5 g/L sodium bicarbonate.

Interferon preparation. Crude extracts of transgenic and wild-type plants were generated using PEB as described above. His-tagged transgenic cpIFN-a2b was purified under native conditions by passing crude extracts over HIS-Select® spin columns (Sigma) as per the manufacturer's protocol. Flow-through, wash and eluate fractions were collected separately for analysis. PEG-Intron was used as the positive control for IFN-a2b activity. We routinely obtained greater than 85% purity of cpIFN-a2b (data not shown).

VSV CPE assay. The Indiana strain of the vesicular stomatitis virus (VSV) was propagated in BHK cells (initial stocks were a kind gift from Dr. Glen Barber, University of Miami). The virus was titered by plaque formation assay in BHK cells. All viral dilutions were made in cell culture medium. BHK cells were seeded in a 96-well plate one day prior to infection at a concentration of 1×105 cells/ml. Cells were treated for six hours prior to infection with either crude extract or purified protein (25 v1), then infected with 3,000 plaque forming units (PFU) of VSV (25 p. 1); control wells were treated either with VSV alone or media alone. The assay was stopped 24 hours later. The medium from each well was aspirated and cells were fixed and stained by adding 100 μl of 0.5% (w/v in 70% methanol) crystal violet solution. After one minute, the crystal violet solution was decanted and the wells were gently rinsed with water. After drying, the wells were examined under an inverted light microscope (Leica Microsystems, Wetzlar, Germany) and photographs taken with a digital camera (Olympus America, Melville, N.Y.). The emission spectra of the crystal violet-stained wells were determined in a microplate reader at 570 nm.

Titering the interferon. The biological activity of the various preparations of interferon was calculated based on that of the PEG-Intron standards and expressed in international units (IU). The mock controls—no interferon, no VSV—were assigned protection values of 0% and 100%, respectively. A formula was generated that correlated the optical density (OD) readings at 570 nm with the percent protection of the mocks. Then, using the calculated biological activity of PEG-Intron (7×10⁷ IU/mg), a second formula correlating the percent protection with the standardized IU of PEGIntron was generated and used to calculate the biological activity of all experimental samples.

HIV-1 inhibition assay. The HIV-1 laboratory strains BaL (R5) and IIIB (X4) were obtained from the NIH AIDS Research and Reference Reagent Program. HIV-1 BaL was propagated in PM1 cells over 16 days. Supernatants containing virus were collected every other day starting 5 days after infection, passed through a 0.45 μm pore size filter and stored in aliquots at −80° C. HIV-1 IIIB was similarly propagated using H9 cells. Virus was quantitated by ELISA for p24^{gag} (PerkinElmer, Boston, Mass.).

The protocol for this assay is described elsewhere (Venkataraman et al. 2005). TZM-BL cells, also known as JC53-BL cells (Wei et al., 2002), which contain a luciferase expression cassette driven by the HIV LTR, were seeded in 96-well dishes (at about 4,000 cells/well). After 24 hours, cells were treated in triplicate with 50 μl of culture medium containing various preparations of IFN-a2b or vehicle control (PBS). Culture medium or virus diluted in culture medium (2 ng/ml p24 for BaL and 5 ng/ml p24 for IIIB) in 50 μl was immediately added to each well and allowed to incubate at 37° C. in 5% CO₂ for 24 hours. Luciferase activity was subsequently measured with Bright-Glo™ reagents (Promega, Madison. Wis.) according to the manufacturer's instructions using an LMax luminometer (Molecular Devices, Sunnyvale, Calif.). Cytotoxicity and the metabolic activity of the cells were verified by a tetrazolium-based (MIT) assay according to the manufacturer's instructions (R&D Systems, Minneapolis, Minn.).

### In Vivo Assays to Study Functionality of Interferon in Mice.

Mice. Five-week old C57BL/6 mice were purchased from Charles River Laboratories (Wilmington, Mass.). Groups of mice were treated either with PBS (3 mice), PEG-Intron (5 mice). or cpIFN-a2b (5 mice) purified from transgenic tobacco leaves. Mice were treated with 20.000 IU of cpIFN-a2b diluted in 100 μl PBS. Biological activity was based on the performance of purified protein in the VSV CPE assay. The doses were administered daily for three consecutive days via intraperitoneal injection using a tuberculin syringe fitted with a 27-gauge needle. Mice were sacrificed 48 hours after the final dose by carbon dioxide inhalation.

Reagents. All antibodies used for flow cytometry and primary antibodies for immunohistochemistry were obtained from BD Biosciences (San Jose, Calif.); conjugated antibodies for immunohistochemistry were obtained from Molecular Probes (Eugene, Oreg.).

Collection of tissue samples. Upon sacrifice, mice were placed supine and the abdomen opened along the ventral midline. A 23-gauge butterfly needle was placed in the right ventricle and sterile PBS was injected into the mouse for 5 minutes. One-half of the spleen was excised and immediately placed in cold culture medium (RPMI 1640 supplemented with 10% FBS). A solution of 4% paraformaldehyde was then injected into the mouse via the right ventricle for 15 minutes. The remaining portion of the spleen was then excised and placed sequentially in 4% PFA, 10% sucrose, 20% sucrose, and 30% sucrose. each for a period of 18-24 hours. Samples were then flash frozen in Tissue Freezing Medium (Triangle Biomedical Sciences, Durham, N.C.) and flash frozen in 2-methylbutane (Sigma) over liquid nitrogen as fixed tissue specimens.

Immunohistochemistry. Sections of fixed frozen tissue (10 μm) were taken on a cryostat slicer (Microm HM505E. Mikron Instruments, San Markos, Calif.) at −20° C. Sections were mounted on Superfrost/Plus microscope slides (Fisher Scientific, Fair Lawn, N.J.). Slides were washed 3 times with PBS, then blocked with PBS containing 10% BSA and 0.3% Triton X-100® for 1 hour at RT. Sections were stained with one of the following primary antibodies (all antibodies were diluted 1:250 in PBS containing 1% BSA and 0.3% Triton X-100®): mouse anti-H-2 Kb (clone AF6-88.5), rat anti-CD49b (clone DX5), or mouse anti-NK1.1 (clone PK136). Sections were incubated with primary antibody overnight at 4° C. Slides were washed three times and then incubated with either of the following tagged secondary antibodies: rabbit anti-mouse/Alexa 488 or goat anti-rat Alexa 555. Sections were incubated with primary antibody for 2 hours at RT, in the dark. Slides were washed three times; if double staining was required, the sequence of primary antibody staining, wash, secondary antibody staining, and wash was repeated. At the end of the staining procedure, slides were fixed by adding 50 of VectaShield® Mounting Medium with DAPI (Vector Laboratories, Burlingame, Calif.). Coverslips were set and slides were analyzed on a Leica DM4000B fluorescent microscope (Leica Microsystems). For each filter cube used, samples were acquired at the same exposures to equilibrate light input. Then, using software-specific tools, the splenic tissue was isolated and the MFI of the antibody staining was tabulated to account for both positively and negatively stained regions alike (Adobe Systems, San Jose, Calif.).

Ex vivo preparation of splenocytes. Spleen tissue in culture medium was dissociated using the plunger of 10 ml syringe over a 70 μm cell strainer (BD Biosciences). The cells were washed once with PBS containing 2% FBS and pelleted by centrifugation at 1,000 rpm for 10 minutes. The supernatant was aspirated and the cells were resuspended in culture medium at a concentration of about 108 cells/ml.

Immunophenotyping. Splenocytes were analyzed for cell surface expression of the following markers: mouse anti-H-2 K^{b} (clone AF6-88.5) conjugated to fluorescein isothiocyanate (FITC), rat anti-CD49b (clone DX5) conjugated to phycoerythrin (PE), or mouse anti-NK1.1 (clone PK136) conjugated to allophycocyanin (APC). Samples were analyzed on a BD FACSAria® (BD Biosciences).

In conclusion, disclosed herein is an invention in which IFN-a2b has been expressed in tobacco chloroplasts and in transgenic lines grown in the field after obtaining USDA-APHIS approval. Southern blots confirmed stable, site-specific integration of transgenes into chloroplast genomes and homoplasmy in several generations. Western blots detected monomeric and multimeric forms of cpIFN-a2b. ELISA showed up to 20% of total soluble protein or 3 mg IFN per gram of leaf (fresh weight). CpIFN-a2b possesses both in vitro and in vivo biological activity, in both crude extract and purified forms. The induced up-regulation of MHC I molecules and activation of NK cells is consistent with the critical role IFN-a2b plays in early immune responses. MHC I is a necessary component of the antiviral response, increasing the presentation of foreign peptides to circulating immune cells primed for attack. NK cells survey the body for changes in MHC I expression, an indicator of abnormal situations such as cancer. The priming of NK cells mediated by IFN-a2b helps mobilize the immune system and facilitate the clearance of tumors. Our results provide a simple and cost-effective method for producing functional IFN-a2b.

The commercial potential of molecular pharming of plant-made pharmaceuticals has resulted in regulatory agencies formulating guidelines to protect the environment and consumers. Clearly, the rationale and risks of molecular pharming are very different from the first generation of crops genetically modified to enhance agronomic traits. This invention employs the use of chloroplast genetic engineering to avoid the transmission of transgenes via pollen. A non-food/non-feed crop has been used to express the therapeutic protein only in vegetative tissues. In addition, the transgenic plants were harvested before any reproductive structures appeared, thereby eliminating the spread of transgenes via pollen or seeds. This is the first example of a human blood protein produced in the field in transgenic plants. Large-scale production and purification in GMP facilities should lead to human clinical trials and further advance this field.

Accordingly, in the drawings and specification there have been disclosed typical preferred embodiments of the invention and although specific terms may have been employed, the terms are used in a descriptive sense only and not for purposes of limitation. The invention has been described in considerable detail with specific reference to these illustrated embodiments. It will be apparent, however, that various modifications and changes can be made within the spirit and scope of the invention as described in the foregoing specification and as defined in the appended claims.

1. A transgenic plastid containing a genome transformed by integration of an expression cassette carrying a non-plant gene encoding an interferon polypeptide.

2. The plastid of claim 1, wherein the interferon consists of IFNa2b.

3. The plastid of claim 1, wherein said interferon polypeptide is fused with a polyhistidine tag and a thrombin cleavage site.

4. The plastid of claim 1, said plastid being operably contained in a plant cell.

5. The plastid of claim 1, said plastid being operably contained in a plant cell of a Nicotiana species.

6. The plastid of claim 1, said plastid being operably carried in a plant of a Nicotiana species.

7. The plastid of claim 1, said plastid being operably carried in a homoplasmic plant of the genus Nicotiana.

8. The plastid of claim 1, said plastid being operably carried in a homoplasmic plant of genus and species Nicotiana tabaccum.

9. A plant containing a plastid genome transformed by integration of an expression cassette carrying a gene encoding an interferon polypeptide.

10. The plant of claim 9, wherein said plant is homoplasmic for the plastid genome carrying a gene encoding the interferon polypeptide.

11. The plant of claim 9, wherein the interferon polypeptide is fused with a polyhistidine tag and a thrombin cleavage site.

12. An isolated cell of the plant of claim 9.

13. The plant of claim 9, wherein said plant is of the genus Nicotiana.

14. The plant of claim 9, wherein said plant is a cultivar of genus and species Nicotiana tabaccum.

15. The plant of claim 9, wherein said plant is cultivar Petit Havana of genus and species Nicotiana tabaccum.

16. The plant of claim 9, wherein the interferon is IFNa2b.

17. A purified polypeptide of interferon, said polypeptide encoded in a plastid genome and fused with a purification peptide tag and a cleavage site peptide providing a site for cleaving the purification tag so as to result in a functional interferon protein.

18. The polypeptide of claim 17, wherein the interferon polypeptide consists of IFNa2b.

19. The polypeptide of claim 17, wherein the plastid genome is contained in a plant.

20. The plant of claim 19, wherein said plant is of the genus Nicotiana.

21. The plant of claim 19, wherein said plant is a cultivar of genus and species Nicotiana tabaccum.

22. A purified recombinant polypeptide of IFNa2b having a polyhistidine tag and a thrombin cleavage site.

23. The polypeptide of claim 22, wherein said polypeptide is produced in the plant of claim 19.

24. An expression cassette containing a polynucleotide encoding an interferon polypeptide, said expression cassette carried in a vector competent for integrating said expression cassette in a plastid genome.

25. The expression cassette of claim 24, wherein the interferon polypeptide consists of IFNa2b and the cassette polynucleotide includes regions encoding a polyhistidine tag and a thrombin cleavage site fused to said polypeptide

26. An expression cassette containing a polynucleotide encoding an IFNa2b polypeptide, having regions encoding a polyhistidine tag and a thrombin cleavage site fused to said polypeptide and comprising a vector competent for integrating said expression cassette in a plastid genome.
